(19) United States
(12) Patent Application Publication
(10) Pub. No.: US 2026/0168026 A1
(43) Pub. Date: Jun. 18, 2026

(54) CRISPR-CAS-BASED COMPOSITION FOR DETECTION OF LISTERIA MONOCYTOGENES AND LISTERIA MONOCYTOGENES DETECTION METHOD USING SAME

(71) Applicant: PULMUONE CO., LTD., Chungcheongbuk-do (KR)

(72) Inventors: Sang Yong LEE, Cheongju-si Chungcheongbuk-do (KR); Young Min PARK, Cheongju-si Chungcheongbuk-do (KR); Sang Yoon LEE, Cheongju-si Chungcheongbuk-do (KR); Hyo Yul LEE, Cheongju-si Chungcheongbuk-do (KR); Sang Gu KIM, Cheongju-si Chungcheongbuk-do (KR)

(21) Appl. No.: 19/121,041

(22) PCT Filed: Jun. 15, 2023
(86) PCT No.: PCT/KR2023/008318

§ 371 Date: Mar. 5, 2026

(30) Foreign Application Priority Data
Oct. 14, 2022 (KR) 10-2022-0132701

(51) Int. Cl. C12Q 1/6883; C12Q 1/6851

(52) U.S. Cl. CPC C12Q 1/6883; C12Q 1/6851; C12Q 2531/113

(57) ABSTRACT

The present invention relates to a CRISPR-Cas-based composition for detection of Listeria monocytogenes and a Listeria monocytogenes detection method using same and, more specifically, to a composition for detection of Listeria monocytogenes, comprising a primer pair capable of specifically amplifying Listeria monocytogenes by isothermal amplification, a guide RNA, and a CRISPR-Cas protein, and a detection method using same.

### TECHNICAL FIELD

The present application claims priority to Korean Patent Application No. 10-2022-0132701, filed on Oct. 14, 2022, and the entire disclosure of the above application is incorporated herein by reference.

The present invention relates to a composition for detecting Listeria monocytogenes based on CRISPR-Cas and a method for detecting Listeria monocytogenes using the same, and more specifically, to a composition for detecting Listeria monocytogenes comprising a primer pair, a guide RNA, and a CRISPR-Cas protein, which can specifically amplify Listeria monocytogenes by isothermal amplification, and a detection method using the same.

### BACKGROUND OF THE INVENTION

Listeriosis is caused by infection with Listeria monocytogenes and is one of the most important zoonotic diseases. Listeria monocytogenes causes encephalitis, abortion, neonatal septicemia, and central nervous system infections in both humans and animals. Additionally, Listeria monocytogenes is commonly found in soil, manure, spoiled vegetables, or dairy products, and it is relatively heat-resistant and capable of growing at refrigerator temperatures, causing food poisoning through refrigerated and stored foods.

In humans, although other routes such as contact with infected animals or inhalation of contaminated air have occasionally been reported, infection through the gastrointestinal tract after consuming contaminated food is the most significant route of infection. In most cases, infection begins when Listeria bacteria pass through the intestinal mucosa. Even during the process of passing through the stomach, where some bacteria are killed, they reach the intestines. If they survive the antibiotic effects of bile upon reaching the intestines, they attach to the intestinal mucosa and invade inward.

The genus Listeria is divided into seven species: Listeria monocytogenes, Listeria ivanovii, Listeria seeligeri, Listeria innocua, Listeria welshimeri, Listeria grayi, and Listeria murrayi. Among them, the pathogenic species are Listeria monocytogenes and Listeria ivanovii. While Listeria monocytogenes is pathogenic to both humans and animals, Listeria ivanovii is known to infect only hoofed animals such as cattle and sheep. The other species require decayed nutrients such as soil or rotting vegetables.

The standard culture method is typically used as a method for detecting Listeria monocytogenes from various origins. However, this method is time-consuming and has the disadvantage of requiring intensive labor to process large quantities of samples simultaneously. Additionally, this method has the drawback of making it difficult to distinguish Listeria monocytogenes from other Listeria species in Listeria-selective media.

Recently, gene-based detection methods such as PCR (Polymerase Chain Reaction) and real-time PCR have been applied in various fields in combination with the conventional standard culture method. These methods amplify strain-specific genes to detect the causative bacteria, offering the advantage of providing results more simply and quickly compared to the standard culture method. However, in the case of PCR, there is the inconvenience of having to perform electrophoresis on an agar gel to confirm the amplification product. Additionally, there are concerns about the inhibition of the gene amplification reaction by components of food samples such as milk and meat.

### DETAILED DESCRIPTION OF THE INVENTION

### Problem to be Solved

Accordingly, the present inventors have conducted extensive research to solve the aforementioned problems and to develop a method capable of detecting Listeria monocytogenes in food or biological samples very rapidly and accurately. As a result, we discovered that Listeria monocytogenes can be detected with very high sensitivity and specificity through the use of a primer pair consisting of the nucleotide sequences of SEQ ID NOs: 1 and 2, and optionally through a gene-editing reaction using guide RNA and CRISPR-Cas proteins, thereby completing the present invention.

Therefore, an object of the present invention is to provide a primer pair for detecting Listeria monocytogenes, comprising a forward primer consisting of the nucleotide sequence of SEQ ID NO: 1 and a reverse primer consisting of the nucleotide sequence of SEQ ID NO: 2.

Another object of the present invention is to provide a composition for detecting Listeria monocytogenes, comprising the primer pair of SEQ ID NOs: 1 and 2, a guide RNA, and an endonuclease.

Additionally, another object of the present invention is to provide a composition for detecting Listeria monocytogenes, consisting of the primer pair of SEQ ID NOs: 1 and 2, a guide RNA, and an endonuclease.

Furthermore, another object of the present invention is to provide a composition for detecting Listeria monocytogenes, consisting essentially of the primer pair of SEQ ID NOs: 1 and 2, a guide RNA, and an endonuclease.

Another object of the present invention is to provide a kit for detecting Listeria monocytogenes, comprising the composition.

Another object of the present invention is to provide a method for detecting Listeria monocytogenes, comprising the following steps:
a) isolating DNA from a sample;
b) amplifying the DNA using the primer pair; and
c) detecting the amplification product.

Another object of the present invention is to provide the use of the primer pair of SEQ ID NOs: 1 and 2, a guide RNA, and an endonuclease for manufacturing a composition for detecting Listeria monocytogenes.

However, the technical problems of the present invention as described above are exemplary and do not limit the scope of the present invention. Furthermore, other objects and advantages of the present invention will become more apparent from the following detailed description of the invention, claims, and drawings.

### Means for Solving the Problem

To achieve the aforementioned objectives of the present invention, the present invention provides a primer pair for detecting Listeria monocytogenes, comprising a forward primer consisting of the nucleotide sequence of SEQ ID NO: 1 and a reverse primer consisting of the nucleotide sequence of SEQ ID NO: 2.

To achieve another objective of the present invention, the present invention provides a composition for detecting Listeria monocytogenes, comprising the primer pair of SEQ ID NOS: 1 and 2, a guide RNA, and an endonuclease.

Additionally, to achieve another objective of the present invention, the present invention provides a composition for detecting Listeria monocytogenes, consisting of the primer pair of SEQ ID NOs: 1 and 2, a guide RNA, and an endonuclease.

Additionally, to achieve another objective of the present invention, the present invention provides a composition for detecting Listeria monocytogenes, consisting essentially of the primer pair of SEQ ID NOs: 1 and 2, a guide RNA, and an endonuclease.

To achieve another objective of the present invention, the present invention provides a kit for detecting Listeria monocytogenes, comprising the composition.

To achieve another objective of the present invention, the present invention provides a method for detecting Listeria monocytogenes, comprising the following steps:
a) isolating DNA from a sample;
b) amplifying the DNA using the primer pair; and
c) detecting the amplification product.

To achieve another objective of the present invention, the present invention provides the use of the primer pair of SEQ ID NOs: 1 and 2, a guide RNA, and an endonuclease for manufacturing a composition for detecting Listeria monocytogenes.

Unless otherwise defined, all technical and scientific terms used herein have the same meaning as commonly understood by one of ordinary skill in the art. The following references provide one of the general definitions of various terms used in the specification of the present invention: Singleton et al., DICTIONARY OF MICROBIOLOGY AND MOLECULAR BIOLOGY (2nd ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY.

Hereinafter, the present invention will be described in detail.

The present invention provides a primer pair for detecting Listeria monocytogenes, comprising a forward primer consisting of the nucleotide sequence of SEQ ID NO: 1 and a reverse primer consisting of the nucleotide sequence of SEQ ID NO: 2.

[SEQ ID NO: 1]
TTACTCGTAGCCATTTCAGCGAGCGGCTTAAT
[SEQ ID NO: 2]
CTTCTTGGCGTCGAGGCATTTATTTCTGGTAT

In the present invention, the term “primer” refers to a short nucleic acid sequence having a free 3′ hydroxyl group that can form base pairs with a complementary template of Listeria monocytogenes nucleic acid and functions as a starting point for template strand replication.

The primer pair can specifically amplify the genes of Listeria monocytogenes, including serotypes 1/2a, 1/2b, 1/2c, and 4b.

The amplification can be performed by any method used in the art to amplify a target (e.g., gene), for example, by recombinase polymerase amplification (RPA), but is not limited thereto.

The amplification product can be, for example, an amplicon, but is not limited thereto.

In the present specification, the term “recombinase polymerase amplification (RPA)” refers to a technique capable of amplifying DNA and RNA, which, unlike conventional PCR, uses DNA-binding proteins and recombinase to rapidly and accurately amplify a target sequence. RPA is similar to conventional PCR but has the advantage of amplifying specific genes under isothermal conditions without temperature changes, thereby reducing reaction time, requiring no special equipment, and enabling cost-effective and portable on-site testing. Recombinase polymerase amplification (RPA) is a method that uses bacteriophage T4 recombinase to denature double-stranded DNA and simultaneously amplifies specific DNA using DNA polymerase and specific primers. Like PCR, it uses a target template and a pair of primers (oligonucleotides) to amplify specific nucleotide sequences of DNA. However, unlike PCR, it induces an amplification reaction under isothermal conditions in the range of 37° C. to 42° C. Currently, RPA has been developed to rapidly produce amplification products, which, along with the advantages of isothermal conditions, is widely recognized and applied in various methods for detecting specific pathogens through specific gene amplification.

The length of RPA amplification products is recommended not to exceed 500 nucleotides (nt) and generally ranges between 100 and 250 nt. The length of RPA primers is recommended to be at least 30 nucleotides, unlike PCR, and is generally 32 to 35 nt. Additionally, unlike PCR, RPA primers are not sensitive to the GC content (GC %) and Tm (melting temperature) value, so the primers should converge to GC % (20-70%) and Tm (50-100° C.) (Analyst, 2019, 144, 31).

The present invention also provides a composition for detecting Listeria monocytogenes, comprising the primer pair of SEQ ID NOs: 1 and 2, a guide RNA, and an endonuclease.

In the present invention, the guide RNA may be crRNA or gRNA. crRNA is referred to as CRISPR RNA. Additionally, gRNA refers to guide RNA. crRNA and gRNA may be single-stranded RNA. Furthermore, crRNA can bind to tracrRNA to activate CRISPR-associated proteins and may be used in a form bound to tracrRNA. In this case, crRNA may have a sequence complementary to a gene sequence specifically present in Listeria monocytogenes.

Additionally, gRNA can bind to a gene sequence specifically present in Listeria monocytogenes to activate CRISPR-associated proteins. The crRNA or gRNA may be RNA consisting of 15 to 40 nucleotides. In one specific example, the crRNA or gRNA may consist of 23 nucleotides. Furthermore, crRNA or gRNA may include additional sequences at the 3′ end to activate CRISPR-associated proteins such as Cas9, Cas12, or Cas13. In one embodiment, the gRNA may consist of SEQ ID NO: 3 or a complementary nucleotide sequence thereof.

[SEQ ID NO: 3]
UUAUAUAGUUCUAGUUUGAUCUUG

In the present invention, the endonuclease may be a CRISPR-associated protein. The term “CRISPR-associated protein” as used in the present invention refers to an enzyme capable of recognizing and cleaving nucleic acids such as DNA or RNA, in case that the nucleic acids contain double-stranded or single-stranded (dsDNA/RNA and ssDNA/RNA). Specifically, these enzymes can recognize and cleave double-stranded or single-stranded nucleic acids bound to crRNA or gRNA.

In one specific example of the present invention, the endonuclease may be activated upon recognizing that the gRNA is bound to the target site. Additionally, upon activation of the endonuclease function, it may exhibit exonuclease activity capable of nonspecifically cleaving double-stranded and/or single-stranded DNA and/or RNA. Furthermore, CRISPR-associated proteins such as Cas12a may exhibit nonspecific exonuclease activity once activated. In such cases, DNA and RNA can be nonspecifically cleaved.

Therefore, in one specific example of the composition of the present invention, Listeria monocytogenes can be detected by the fluorescence emitted from a reporter conjugated to a fluorescent substance, which is cleaved by a nonspecific nuclease activated by the binding of crRNA or gRNA to a specific target site of Listeria monocytogenes, more specifically the lmo0161 gene.

Specifically, one embodiment of the CRISPR-associated protein may be any one selected from the group consisting of Cas1, Cas1B, Cas2, Cas3, Cas4, Cas5, Cas6, Cas7, Cas8, Cas9, Cas10, Cas12a, Cas12b, Cas12c, Cas12d, Cas12e, Cas12g, Cas12h, Cas12i, Cas13a, Cas13b, Cas13c, Cas13d, Cas14, Csy1, Csy2, Csy3, Cse1, Cse2, Csc1, Csc2, Csa5, Csn2, CsMT2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, preferably any one selected from the group consisting of Cas9, Cas10, Cas12a, Cas12b, Cas12c, Cas12d, Cas12e, Cas12g, Cas12h, Cas12i, Cas13a, Cas13b, Cas13c, and Cas13d, more preferably any one selected from the group consisting of Cas12a, Cas12b, Cas12c, Cas12d, Cas12e, Cas12g, Cas12h, and Cas12i, and most preferably Cas12a (Cpf1).

In one aspect of the present invention, the composition may be characterized by further comprising a reporter gene.

In the present invention, it is preferable that the reporter gene is conjugated with a fluorescent substance, and the fluorescent substance may be selected from the group consisting of FAM, TET, JOE, YAKYE, HEX, CY3, ATTO550, TAM, ROX, TxRed, CY35, LC610, LC640, ATTO647N, CY5, VIC, evergreen dye, and ATTO680, but is not limited thereto.

In one aspect of the present invention, the composition may be characterized by being used in a method for detecting Listeria monocytogenes by isothermal amplification reaction and subsequent CRISPR-mediated reaction.

Specifically, if a target site exists in the amplification product amplified through the primer pair consisting of the nucleotide sequences of SEQ ID NO: 1 and 2, the guide RNA complementarily binds to the target site, and the endonuclease, preferably the CRISPR-associated protein, is activated to cleave the sequence of the reporter gene. As a result, the fluorescent substance conjugated to the reporter gene emits fluorescence, thereby detecting Listeria monocytogenes.

The present invention also provides a kit for detecting Listeria monocytogenes comprising the composition.

The kit may be a kit comprising essential components necessary for performing the detection of Listeria monocytogenes, and in addition to the composition, may further comprise test tubes or other suitable containers, reaction buffers (with varying pH and magnesium concentrations), deoxynucleotides (dNTPs), enzymes such as Taq polymerase, DNase, RNase inhibitors, DEPC-water, and sterile water.

The present invention also provides a method for detecting Listeria monocytogenes comprising the following steps:
a) isolating DNA from a sample;
b) amplifying the DNA using the primer pair; and
c) detecting the amplification product.

In one embodiment, the present invention provides a method for diagnosing and treating listeriosis in a subject comprising the following steps:
i) isolating DNA from a subject;
ii) amplifying the DNA using the primer pair;
iii) detecting the amplification product;
iv) diagnosing listeriosis if the amplification product of step iii) is detected; and
v) administering a therapeutic agent for treating listeriosis or performing surgery to treat the disease in the diagnosed subject.

The methods comprising steps i) to v) are understood in accordance with the methods comprising steps a) to d) described above.

Step v) is a step of treating the disease in the subject diagnosed with the disease in step iv) through administration of therapeutic agents such as antibiotics including ampicillin, gentamicin, tetracycline, or aminoglycosides, or through surgical means.

The term “treatment” in the present invention comprehensively refers to improving the symptoms of listeriosis or listeriosis itself, which may include curing, substantially preventing, or improving the condition, and includes alleviating, curing, or preventing one or more symptoms or most symptoms caused by the disease, but is not limited thereto.

In the present invention, the term “sample” or “subject” refers to a material or organism used for testing, examination, or analysis, and is not particularly limited in type, but may be a food or biological sample suspected of containing Listeria monocytogenes.

In the present invention, the biological sample may include tissues, cells, whole blood, serum, plasma, cerebrospinal fluid, colostrum, synovial fluid, saliva, feces, and cell culture supernatants, but is not limited thereto.

DNA may be obtained from the sample (or subject) using conventional known DNA extraction methods. If the starting material is gDNA, the gDNA may be isolated using conventional methods known in the art, and if the starting material is mRNA, it may be synthesized into cDNA using reverse transcriptase. For example, the DNA isolation is not particularly limited as long as it is a method for isolating genomic DNA (gDNA) from a sample. For instance, commercially available DNA isolation kits such as the DNeasy mini kit from Qiagen may be used.

In the present invention, the term “amplification” may be any one selected from the group consisting of PCR, RT-PCR, and isothermal amplification, and preferably isothermal amplification.

The term “isothermal amplification” refers to a method for amplifying a target nucleic acid sequence (template) under constant reaction temperature conditions, and may be any one selected from the group consisting of recombinase polymerase amplification (RPA), loop-mediated isothermal amplification (LAMP), helicase-dependent amplification (HDA), rolling circle amplification (RCA), multiple displacement amplification (MDA), strand displacement amplification (SDA), and nucleic acid sequence-based amplification (NASBA), and preferably recombinase polymerase amplification (RPA).

The detection of the amplification product may be performed by capillary electrophoresis, DNA chip, gel electrophoresis, radiometric measurement, fluorescence measurement, or phosphorescence measurement, but is not limited thereto.

As one method for detecting the amplification product, for example, capillary electrophoresis may be performed. Capillary electrophoresis may use, for instance, an ABI Sequencer. Additionally, gel electrophoresis may be performed, and depending on the size of the amplification product, agarose gel electrophoresis or acrylamide gel electrophoresis may be used. Furthermore, in the fluorescence measurement method, if Cy-5 or Cy-3 is labeled at the 5′-end of the primer and PCR is performed, the target sequence can be detected with a fluorescent label, and the labeled fluorescence can be measured using a fluorescence detector. In the radiometric measurement method, radioactive isotopes such as 32P or 35S may be added to the PCR reaction mixture during PCR to label the amplification product, and the radioactivity may be measured using radiometric devices such as a Geiger counter or a liquid scintillation counter. Additionally, RPA amplification products may be obtained and separated by methods such as agarose gel electrophoresis, and the presence of DNA with a length corresponding to the DNA polymerized by the primer set used, specifically DNA with a length of 230 bp, may be confirmed to detect Listeria monocytogenes.

In one aspect of the present invention, after step b), the method may further comprise mixing the amplification product with a composition comprising a guide RNA, an endonuclease, and a reporter gene.

In this case, the presence of the Listeria monocytogenes can be confirmed by detecting the fluorescence reaction of the reporter gene according to the gene-editing reaction by the guide RNA and the endonuclease.

The composition comprising the primer pair and the guide RNA provided by the present invention can specifically detect Listeria monocytogenes. Therefore, by using this, Listeria monocytogenes in a biological sample to be diagnosed (e.g., saliva, feces, blood, urine, etc.)

can be rapidly and accurately detected, enabling very fast and accurate diagnosis of diseases caused by Listeria monocytogenes infection, specifically food poisoning, listeriosis, and the like.

The term “diagnosis” as used in the present specification includes determining whether an object currently has a specific disease or condition, or determining the prognosis of an object suffering from a specific disease or condition.

In addition, the present invention provides the use of the primer pair of SEQ ID NOs: 1 and 2, the guide RNA, and the endonuclease for manufacturing a composition for detecting Listeria monocytogenes.

The term “comprising” as used in the present specification is used in the same sense as “including” or “characterized by,” and does not exclude additional components or steps of methods that are not specifically mentioned in the composition or method according to the present invention. Furthermore, the term “consisting of” means excluding additional elements, steps, or components that are not separately described. The term “consisting essentially of” means that, within the scope of the composition or method, it may include substances or steps that do not substantially affect the essential characteristics of the described substances or steps.

### Effect of the Invention

The primer pair provided by the present invention, or the primer pair and the guide RNA, enables the rapid detection of Listeria monocytogenes in a sample with very high sensitivity and specificity.

### BRIEF DESCRIPTION OF THE DRAWINGS

FIG. 1 is a schematic diagram briefly illustrating the gRNA selection process for detecting Listeria monocytogenes.

FIG. 2 is results of performing a gene amplification reaction of Listeria monocytogenes using a combination of RPA primer pairs specific to the target gene of the selected gRNA.

FIG. 3 is results of detecting four serotypes of Listeria monocytogenes and Listeria innocua using the selected gRNA and primer pairs, an endonuclease (CRISPR-Cas12a), and a reporter gene.

FIG. 4 is a table of types of strains used to evaluate detection specificity using the selected gRNA and primer pairs, an endonuclease (CRISPR-Cas12a), and a reporter gene.

FIG. 5 is results of evaluating detection specificity using the selected gRNA and primer pairs, an endonuclease (CRISPR-Cas12a), and a reporter gene.

FIG. 6 is results of evaluating detection sensitivity using the finally selected single gRNA and primer pair, an endonuclease (CRISPR-Cas12a), and a reporter gene.

### DETAILED DESCRIPTION OF THE INVENTION

Hereinafter, preferred embodiments are presented to aid in understanding the present invention. However, the following embodiments are provided merely to facilitate understanding of the present invention, and the content of the present invention is not limited thereto.

### Example 1: Primary Selection of gRNA for Detecting Listeria monocytogenes

The core of research in the diagnostic field using CRISPR gene-editing technology lies in the applied Cas protein and gRNA. In this study, cpf1 (Cas12a), which has activity on dsDNA, was selected, and it is known that this protein requires a T-rich PAM (TTTN) within the target.

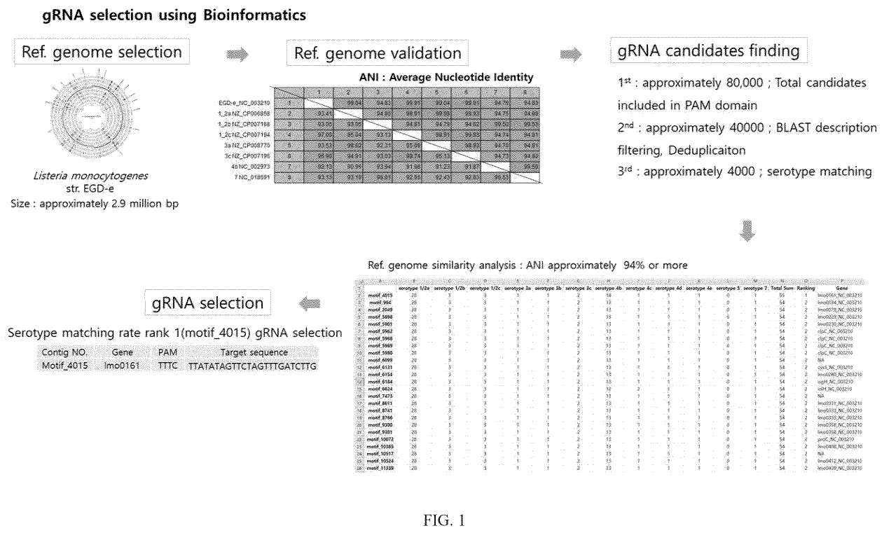

Through bioinformatics, approximately 80,000 gRNA candidates were identified for the Listeria monocytogenes reference genome (NC_003210). Among these, filtering was performed to simultaneously cover four serotypes accounting for over 95% of Listeria monocytogenes clinical outbreaks, narrowing the candidates to approximately 4,000. Subsequently, the gRNA with the highest (rank 1) serotype matching rate (SEQ ID NO: 3 or its complementary sequence) was selected (FIG. 1).

### Example 2: RPA Primer Design

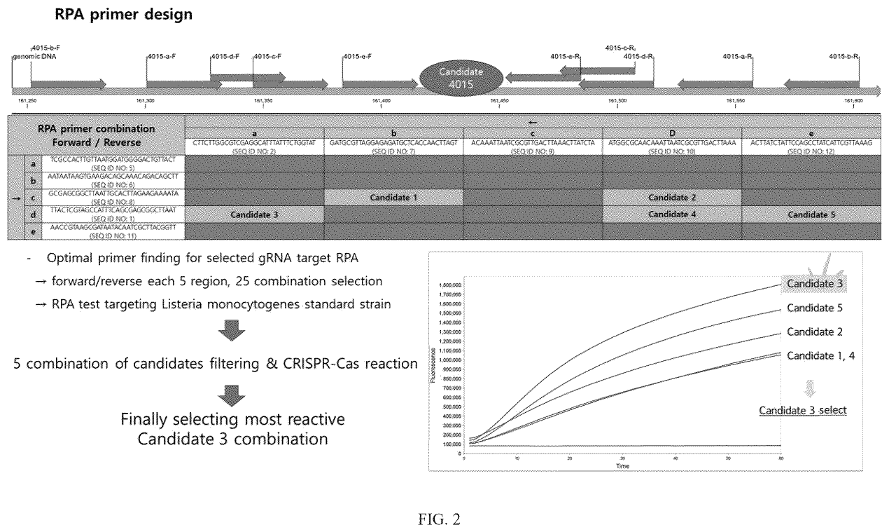

For the gRNA selected in Example 1, optimal RPA primer design was performed by selecting 5 regions for each of the forward and reverse primers, resulting in 25 combinations (FIG. 2). RPA primer validation was conducted on the Listeria monocytogenes standard strain (NCCP 14714), and the primer pair with the best reaction (Forward: SEQ ID NO: 1, Reverse: SEQ ID NO: 2) was finalized (FIG. 2).

### Example 3: Detection Experiment of Listeria monocytogenes Using RPA Primers, gRNA, and CRISPR-Cas12a

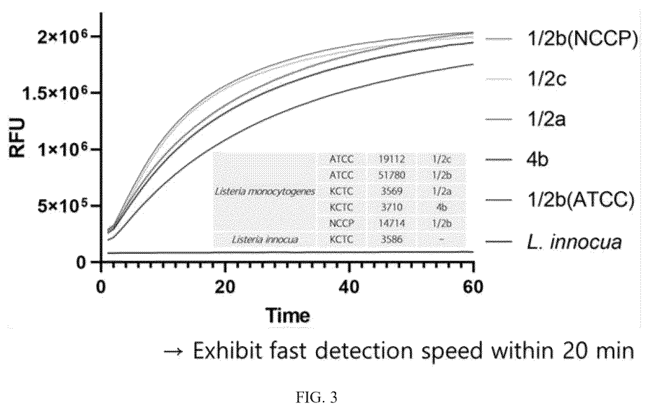

Using the RPA primer pair confirmed to exhibit the best reaction in Example 2, six standard strain extracts shown in FIG. 3 were amplified. The amplified amplicon was subjected to a reaction applying the gRNA and CRISPR-Cas 12a from Example 1, and the detailed reaction conditions are shown in Table 1 below. The dispensed mixture prepared according to the protocol was measured at 1-minute intervals using a 37° C. isothermal fluorescence detector, and the results are shown in FIG. 3.

TABLE 1

| ingredient | Stock conc. | | final concentration(tube) | |
|---|---|---|---|---|
| buffer solution | 10 | x | 1 | x |
| gRNA | 10 | μM | 0.5 | μM |
| Cpf1 | 1 | μM | 00.5 | Mm |
| ssDNA reporter | 10 | μM | 0.5 | μM |
| Rnase inhibitor | 40 | U/μL | 10 | U |

| | |
|---|---|
| distilled water | Add for total amount of reaction to |
| | become 18 μL |
| RPA amplicon | Add 2 μL |

* ssDNA reporter: F-TTTTTTTT-Q

As shown in FIG. 3, the detection reaction for Listeria monocytogenes using the primer pair selected in Example 2 and the gRNA designed in Example 1 was confirmed to exhibit a fast detection speed and high fluorescence values.

### Example 4: Evaluation of Detection Specificity of RPA Primers and gRNA

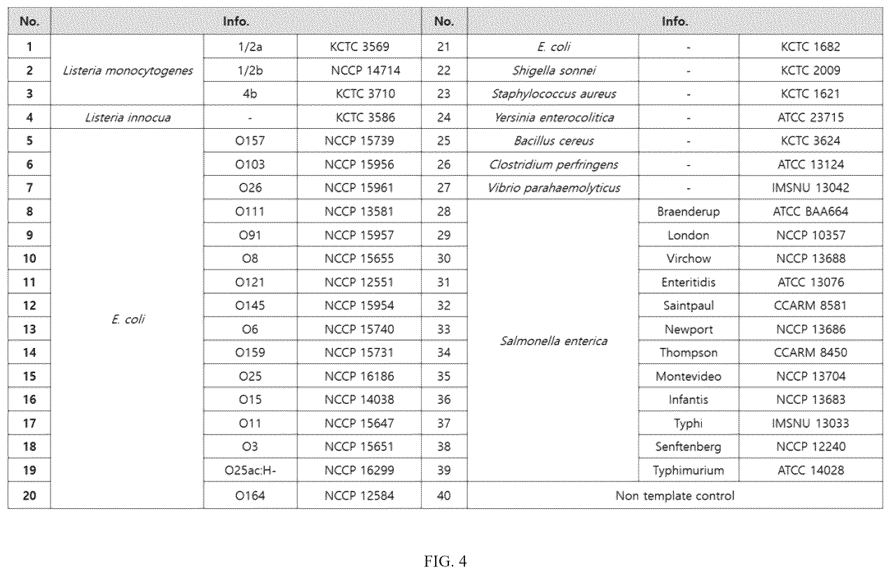

To verify whether the gRNA and RPA primer pair produced in Examples 1 and 2 can specifically detect only Listeria monocytogenes, DNA extraction was performed on 36 standard strains other than Listeria monocytogenes (FIG. 4). Amplification was then conducted using the RPA primer pair produced in Example 2, and a reaction mixture was prepared by applying the gRNA and CRISPR-Cas12a produced in Example 1 according to the protocol in Table 1. A 37° C. isothermal amplification reaction was performed, and fluorescence was observed at the endpoint (60 minutes).

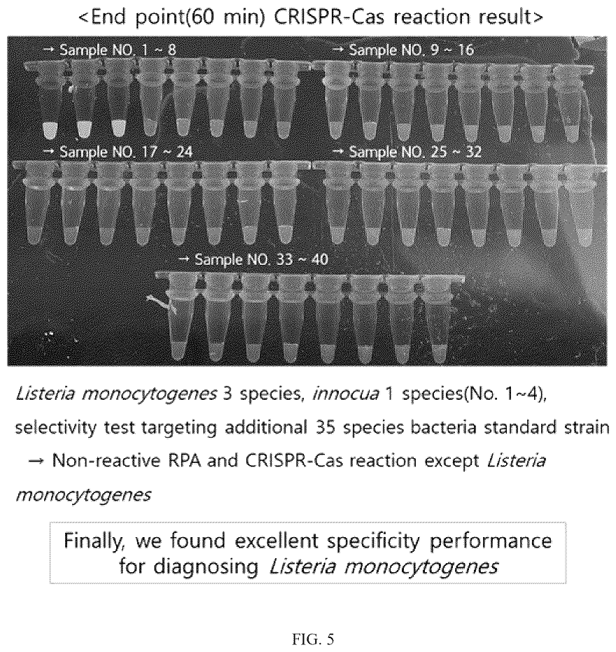

As shown in FIG. 5, the detection reaction using the gRNA, RPA primer pair, and CRISPR-Cas12a according to the present invention was confirmed to specifically detect only Listeria monocytogenes.

### Example 5: Evaluation of Detection Sensitivity of RPA Primers and gRNA

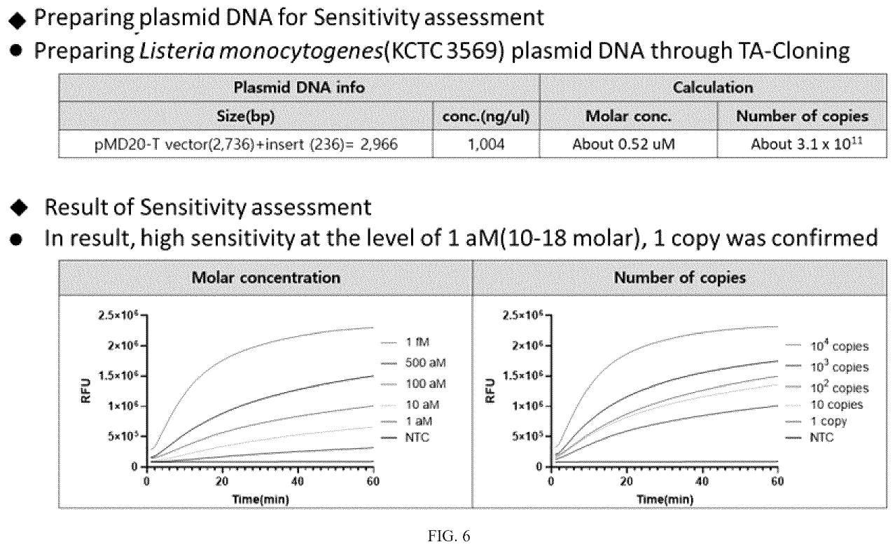

To verify the sensitivity of the gRNA and RPA primer pair produced in Examples 1 and 2 for Listeria monocytogenes, amplicons for the Listeria monocytogenes standard strain (NCCP 14714) were obtained through PCR using the RPA primer pair. Standard plasmid DNA was then prepared using the obtained amplicon (FIG. 6). The prepared plasmid DNA was serially diluted to molar concentrations and copy levels of 1 fM, 500 aM, 100 aM, 10 aM, 1 aM, and 104 copies, 103 copies, 102 copies, 10 copies, and 1 copy, respectively. Detection sensitivity evaluation was conducted using the RPA primer pair, gRNA, and CRISPR-Cas12a, and the results are shown in FIG. 6.

As shown in FIG. 6, it was confirmed that detection is possible with high sensitivity at the level of 1 aM (10-18 molar) and 1 copy.

### INDUSTRIAL APPLICABILITY

The primer pair provided by the present invention, or the use of the primer pair and the guide RNA, enables the rapid detection of Listeria monocytogenes in a sample with very high sensitivity and specificity, thereby having very high industrial applicability.

1.-17. (canceled)

18. A composition for detecting Listeria monocytogenes, comprising a primer pair capable of amplifying the sequence TTATATAGTTCTAGTTTGATCTTG (SEQ ID NO: 4) within Listeria monocytogenes lmo0161 gene, a guide RNA comprising the nucleotide sequence SEQ ID NO: 3, and an endonuclease, wherein the endonuclease is selected from the group consisting of Cas12, Cas13 and Cas14.

19. The composition according to claim 18, wherein the primer pair comprises a forward primer consisting of the nucleotide sequence of SEQ ID NO: 1 and a reverse primer consisting of the nucleotide sequence of SEQ ID NO: 2.

20. The composition according to claim 18, wherein the endonuclease is selected from the group consisting of Cas12a, Cas12b, Cas12c, Cas12d, Cas12e, Cas12g, Cas12h, Cas12i, Cas13a, Cas13b, Cas13c, Cas13d, and Cas14.

21. The composition according to claim 18, further comprising a reporter gene.

22. The composition according to claim 21, wherein the reporter gene is conjugated with a fluorescent substance.

23. The composition according to claim 22, wherein the fluorescent substance is one or more substance selected from the group consisting of FAM, TET, JOE, YAKYE, HEX, CY3, ATTO550, TAM, ROX, TxRed, CY35, LC610, LC640, ATTO647N, CY5, VIC, evergreen dye, and ATTO680.

24. The composition according to claim 18, wherein the composition detects Listeria monocytogenes by isothermal amplification reaction and subsequent CRISPR-mediated reaction.

25. A kit for detecting Listeria monocytogenes, comprising the composition according to claim 18.

26. A method for detecting Listeria monocytogenes, comprising the steps of:
(a) isolating nucleic acid from a sample;
(b) amplifying the nucleic acid using a primer pair capable of amplifying the sequence TTATATAGTTCTAGTTTGATCTTG (SEQ ID NO: 4) within Listeria monocytogenes lmo0161 gene or its complementary sequence, and mixing the amplification product with a composition comprising a guide RNA, an endonuclease, and a reporter gene, wherein the endonuclease is selected from the group consisting of Cas12, Cas13 and Cas14; and
(c) detecting the amplification product.

27. The detection method according to claim 26, wherein the amplification is isothermal amplification.

28. The detection method according to claim 27, wherein the isothermal amplification is recombinase polymerase amplification (RPA).

29. The detection method according to claim 26, wherein the sample is a food or biological sample.


SEQUENCE LISTING (12 sequences)

SEQ ID NO: 1 - DNA, 32 bp, synthetic construct
  source 1..32: mol_type = other DNA
ttactcgtagccatttcagcgagcggcttaat

SEQ ID NO: 2 - DNA, 32 bp, synthetic construct
  source 1..32: mol_type = other DNA
cttcttggcgtcgaggcatttatttctggtat

SEQ ID NO: 3 - RNA, 24 nt, synthetic construct
  source 1..24: mol_type = other RNA
ttatatagttctagtttgatcttg

SEQ ID NO: 4 - DNA, 24 bp, synthetic construct
  source 1..24: mol_type = other DNA
ttatatagttctagtttgatcttg

SEQ ID NO: 5 - DNA, 32 bp, synthetic construct
  source 1..32: mol_type = other DNA
tcgccacttgttaatggatggggactgttact

SEQ ID NO: 6 - DNA, 32 bp, synthetic construct
  source 1..32: mol_type = other DNA
aataataagtgaagacagcaaacagacagctt

SEQ ID NO: 7 - DNA, 32 bp, synthetic construct
  source 1..32: mol_type = other DNA
gatgcgttaggagagatgctcaccaacttagt

SEQ ID NO: 8 - DNA, 32 bp, synthetic construct; identical to SEQ ID NO: 6
  source 1..32: mol_type = other DNA

SEQ ID NO: 9 - DNA, 32 bp, synthetic construct
  source 1..32: mol_type = other DNA
acaaattaatcgcgttgacttaaacttatcta

SEQ ID NO: 10 - DNA, 32 bp, synthetic construct
  source 1..32: mol_type = other DNA
atggcgcaacaaattaatcgcgttgacttaaa

SEQ ID NO: 11 - DNA, 32 bp, synthetic construct
  source 1..32: mol_type = other DNA
aaccgtaagcgataatacaatcgcttacggtt

SEQ ID NO: 12 - DNA, 32 bp, synthetic construct
  source 1..32: mol_type = other DNA
acttatctattccagcctatcattcgttaaag